(19) United States
(12) Patent Application Publication
(10) Pub. No.: US 2022/0143252 A1
(43) Pub. Date: May 12, 2022

(54) MANAGEMENT SYSTEM FOR MANAGING STERILIZATION PROCESS RECORD, INFORMATION PROCESSING APPARATUS, MANAGEMENT METHOD, AND COMPUTER-READABLE STORAGE MEDIUM

(71) Applicant: CANON KABUSHIKI KAISHA, Tokyo (JP)

(72) Inventors: Hiroki Kato, Shizuoka (JP); Motohiro Furusawa, Shizuoka (JP); Kazuhide Okuno, Shizuoka (JP)

(21) Appl. No.: 17/432,468

(22) PCT Filed: Feb. 19, 2020
(86) PCT No.: PCT/JP2020/006592

§ 371 Date: Aug. 19, 2021

(30) Foreign Application Priority Data
Feb. 27, 2019 (JP) 2019-034745
Jul. 16, 2019 (JP) 2019-131401

(51) Int. Cl. A61L 2/28; A61L 2/24; G01N 21/78; G16H 40/20

(52) U.S. Cl. CPC A61L 2/28; A61L 2/24; A61L 2202/14; G16H 40/20; G01N 21/78

(57) ABSTRACT

A management system that manages records of sterilization processes performed by a sterilization processing apparatus on a set including one or more sterilization targets and a first indicator including a discoloration area whose color changes according to an achievement degree of a sterilization process is provided. The management system includes: retaining means for retaining sterilization record information including the achievement degree of the sterilization process determined based on a color of the discoloration area of the first indicator of the set, and placement information regarding placement of the set to the sterilization processing apparatus, with respect to each sterilization process performed on the set; and generating means for generating information for determining a relationship between the placement information and the achievement degree of the sterilization process based on the sterilization record information.

### TECHNICAL FIELD

The present invention relates to a technique of managing records of sterilization processes performed on sterilization targets.

### BACKGROUND ART

Sterilization processes are performed on sterilization targets such as medical instruments in a hospital. On this occasion, so-called sterilization indicators such as a chemical indicator (hereinafter, CI) are used in order to determine the achievement degree of the sterilization process performed on the sterilization targets. The CI includes a discoloration area whose color changes according to the achievement degree of a condition needed for the sterilization process in which sterilizing agents (such as vapor or hydrogen peroxide) are used. Japanese Patent No. 3414291 discloses a management apparatus that manages records of the sterilization process. According to Japanese Patent No. 3414291, the date and time of a process of sterilizing a sterilization target, the sterilization condition, the CI determination result are recorded.

In recent years, emphasis is on keeping the quality of the sterilization process at a high level. Here, the quality of the sterilization process may change due to various factors. Therefore, the quality of the sterilization process cannot be kept at a high level by simply recording the date and time of the sterilization process performed on a sterilization target, the sterilization condition, and the determination result determined using a CI.

### SUMMARY OF INVENTION

According to an aspect of the present invention, a management system that manages records of sterilization processes performed by a sterilization processing apparatus on a set including one or more sterilization targets and a first indicator including a discoloration area whose color changes according to an achievement degree of a sterilization process is provided. The management system includes: retaining means for retaining sterilization record information including the achievement degree of the sterilization process determined based on a color of the discoloration area of the first indicator of the set, and placement information regarding placement of the set to the sterilization processing apparatus, with respect to each sterilization process performed on the set; and generating means for generating information for determining a relationship between the placement information and the achievement degree of the sterilization process based on the sterilization record information.

Further features of the present invention will become apparent from the following description of exemplary embodiments with reference to the attached drawings.

### BRIEF DESCRIPTION OF DRAWINGS

FIG. 1 is a diagram illustrating a sterilization workflow according to one embodiment.

FIG. 2 is a diagram illustrating a configuration of a management system according to one embodiment.

FIG. 3 is a diagram illustrating a CI according to one embodiment.

FIG. 4 is a cross-sectional view of a measurement apparatus according to one embodiment.

FIG. 5 is a cross-sectional view of a measurement apparatus according to one embodiment.

FIG. 6 is a cross-sectional view of a measurement apparatus according to one embodiment.

FIG. 7 is a cross-sectional view of a measurement apparatus according to one embodiment.

FIG. 8 is a diagram illustrating a state in which the inside of a sterilization processing apparatus according to one embodiment is divided into zones.

FIGS. 9A to 9C are diagrams illustrating a management information table according to one embodiment.

FIG. 10 is a diagram illustrating a graph presented by the management system according to one embodiment.

FIG. 11 is a diagram illustrating a graph presented by the management system according to one embodiment.

FIG. 12 is a diagram illustrating a graph presented by the management system according to one embodiment.

FIG. 13 is a diagram illustrating a management information table according to one embodiment.

FIG. 14 is a diagram illustrating a graph presented by the management system according to one embodiment.

FIG. 15 is a diagram illustrating a graph presented by the management system according to one embodiment.

FIG. 16 is a diagram illustrating a management information table according to one embodiment.

FIG. 17 is a diagram illustrating a placement of sets in the sterilization processing apparatus according to one embodiment.

FIG. 18 is a diagram illustrating a management information table according to one embodiment.

FIG. 19 is a diagram illustrating a relationship between a pair of types of adjacent sets and an achievement degree according to one embodiment.

FIG. 20 is a diagram illustrating a relationship between a combination of types of sets and an achievement degree according to one embodiment.

FIG. 21 is a diagram illustrating a CI according to one embodiment.

FIG. 22 is a diagram illustrating a management information table according to one embodiment.

FIG. 23 is a functional block diagram of the management system according to one embodiment.

FIG. 24 is a functional block diagram of an information processing apparatus according to one embodiment.

### DESCRIPTION OF EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

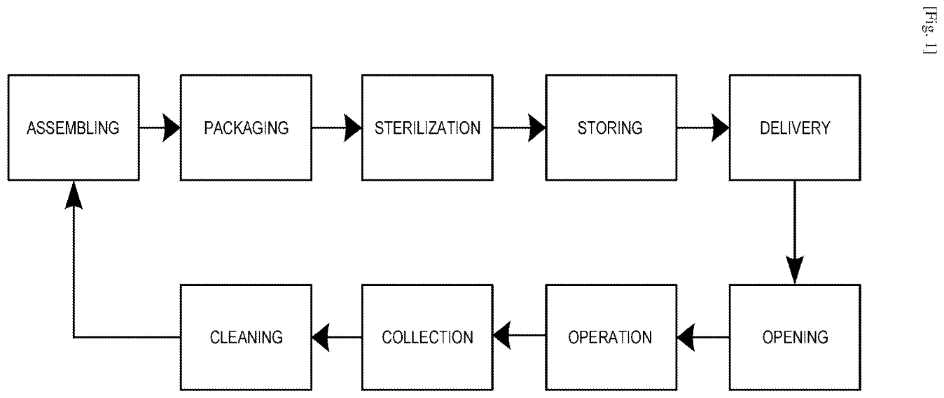

FIG. 1 shows a sterilization workflow relating to a sterilization process according to the present embodiment. In the present embodiment, the sterilization workflow means a series of process stages that are to be performed relating to a sterilization process with respect to a sterilization target. An assembly stage is a stage in which one or more sterilization targets are grouped in a sterilization station. Note that the number of sterilization targets included in one group may be one. The grouping is performed based on a correspondence relationship between operations in which the sterilization targets are used and sterilization processing apparatuses that are used in sterilization processes of the sterilization targets, for example. For example, the sterilization targets that are to be used in the same operation, and on which the sterilization processes are performed by the same sterilization processing apparatus may be grouped into the same group. A packaging stage is a stage in which one set is formed by packaging, in a sterilization station, one or more sterilization targets of one group and a CI (first indicator) using one packaging member. Usually, in hospitals or the like, a plurality of sterilization targets of the same type are used, and therefore a plurality of sets each including the same sterilization target(s) may be formed. Hereinafter, a plurality of sets including the same sterilization target(s) is referred to as a set of the same type. Conversely, the types of two sets being different means that at least one of the sterilization targets included in the respective two sets is different. The type of the CI to be packaged into a set in the packaging stage is determined in accordance with the combination of the sterilization target in the set and a sterilization processing apparatus that performs the sterilization process on the set, or the like. A sterilization stage is a stage in which a sterilization process is performed on a packaged set using a sterilization processing apparatus.

A storing stage is a stage in which a set on which the sterilization process has been performed in the sterilization stage is moved to and stored in a storage cabinet. A delivery stage is a stage of moving a set to be used in an operation or the like from the storage cabinet to a place of usage such as an operation room. An opening stage is a stage of opening the package of a set that has been moved to the place of usage. An operation stage is also a using stage that uses the sterilization target. Note that the achievement degree of the sterilization process is determined using the CI that is enclosed in the set before using the sterilization target in the opening stage, the operation stage, or both of the stages. Then, only when the achievement degree of the sterilization process has been determined to be sufficiently high using the CI, the sterilization target is actually used. A collection stage is a stage in which, after a sterilization target is used, the sterilization target is moved to the sterilization station. A cleaning stage is a stage in which a sterilization target is cleaned in the sterilization station. After a set is cleaned in the cleaning stage, the set is transferred to the assembly stage.

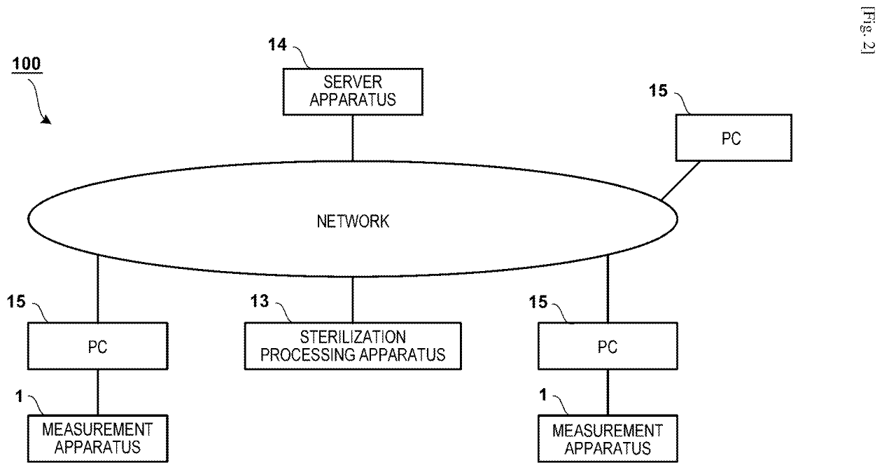

FIG. 2 is a diagram illustrating a configuration of a management system 100 that manages the sterilization workflow. A server apparatus 14, a plurality of personal computers (hereinafter. PCs) 15, and a sterilization processing apparatus 13 are connected to a network. The server apparatus 14, the PCs 15, and the sterilization processing apparatus 13 can communicate to each other via the network. The server apparatus 14 manages management information (sterilization record information) regarding the sterilization workflow. The PCs 15 can add information to the management information retained by the server apparatus 14, modify the retained information, and the like via the network. Also, the PCs 15 and the server apparatus 14 can present various types of information to a user of the management system 100 based on the management information retained by the server apparatus 14. The presentation of various types of information is performed with any method such as displaying presentation contents in a display of a PC 15, outputting the presentation contents to a primer connected to the PC 15, and outputting information indicating the presentation contents to an unshown PC used by the user, for example. In this way, the mode for presenting/displaying various types of information to the user includes various methods, but are collectively expressed as “management system 100 presenting/displaying (information to a user)”, in the following. Note that, in the present invention, the user means a person related to the sterilization workflow such as operators that actually perform work in the respective stages in the sterilization workflow, and a supervisor that supervises the operators.

The PCs 15 are installed at places where, in the sterilization workflow, management information needs to be referred to, information needs to be added to the management information, and the management information needs to be corrected. Also, as shown in FIG. 2, some PCs 15 are connected to respective measurement apparatuses 1. Each measurement apparatus 1 is an apparatus that measures a discoloration area of a CI. Note that, in the present embodiment, the measurement apparatus 1 is configured to also function as a reader apparatus that reads identification information of the CI in addition to the measurement of the discoloration area of the CI. Therefore, the measurement apparatuses 1 are also installed, in addition to the places where a CI needs to be measured, at places where the identification information of the CI needs to be read, and are each connected to a PC 15 installed at the same place. Note that the network in FIG. 2 is not limited to a closed network in a hospital. For example, the sterilization station may be located outside the hospital, and in this case, the network connects the hospital and the sterilization station outside the hospital. Also, the network may be a closed network such as a local area network (LAN) that is not connected to another network, or a network including the LAN and the Internet. For example, the network can be constituted by the Internet and a LAN inside a hospital to be connected the Internet, and in this case, the management system 100 can be configured by installing the server apparatus 14 outside the hospital and connecting it to the Internet.

Also, in the present embodiment, the sterilization processing apparatus 13 is connected to the network, and the PCs 15 and the server apparatus 14 can acquire information to be added to the management information and information for modifying the management information from the sterilization processing apparatus 13. However, the configuration may also be such that the sterilization processing apparatus 13 is not connected to the network. In this case, an operator performs addition, modification, or the like of the management information by operating a PC 15 based on information output from the sterilization processing apparatus 13. Note that, in order for an operator to perform input, update, display, or the like of the management information, the operator needs to operate a PC 15 or the like, the description of operating a PC 15 will be omitted in the following in order to simplify the description. For example, an operator inputting information to the management system 100 by operating a PC 15 is also represented as an operator inputting information to the management system 100.

In the present embodiment, the sets am each given identification information. The identification information may be in the form of a serial number or a bar code that is printed on a seal, for example. Alternatively, the identification information may be recorded in an IC tag. A medium including the identification information may be attached to an exterior face of a packaging member of the set, or a sterilization target of the set, for example. Alternatively, the medium may be attached to a CI enclosed in the set. Also, when a serial number or the like is recorded in a CI itself, the serial number of the CI to be enclosed in a set can be used as the identification information of the set. In the present embodiment, the serial number of a CI enclosed in a set is used as the identification information of the set.

FIG. 3 shows a CI according to the present embodiment. The CI is a sheet-like test paper and includes a discoloration area 21 in which a chemical treatment is performed on its surface. The discoloration area 21 changes its color according to the achievement degree of a sterilization process. In the present embodiment, it is assumed that the discoloration area 21 of a CI is configured such that its color value is a color value C #1 in an initial state, changes to a color value C #2, . . . , and to a color value C #9 according to the achievement degree of the sterilization process, and finally stabilizes at a color value C #10. Hereinafter, the achievement degree of the sterilization process at which a color value C #k (k is an integer from 1 to 10) is obtained is defined as an achievement degree #k. Note that the discoloration area 21 does not take one of the color values C #1 to C #10, and continuously change its color from the color value C #1 to the color value C #10. That is, the discoloration area 21 can take a color between a color value C #m (m is an integer from 1 to 9) and a color value C #m+1. In the present embodiment, the level of the achievement degree of the sterilization process is evaluated in nine stages. Specifically, if the color of the discoloration area 21 is in a range between a color value C #m and a color value C #m+1, the level of the sterilization process is determined to be a level #m. Also, it is assumed that levels #4 to #9 are OK levels and levels #1 to #3 are NG levels, for example. Here, the level of the sterilization process being a level #m means that the achievement degree of the sterilization process is in a range from an achievement degree #m to an achievement degree #m+1.

Also, the CI includes an identification portion 22 having identification information. In FIG. 3, the identification portion 22 is a portion in which a serial number of the CI is printed on a substrate of the CI. Note that the identification portion 22 may also be a portion in which a bar code is printed or an IC tag (wireless tag) attached to the CI, as described above.

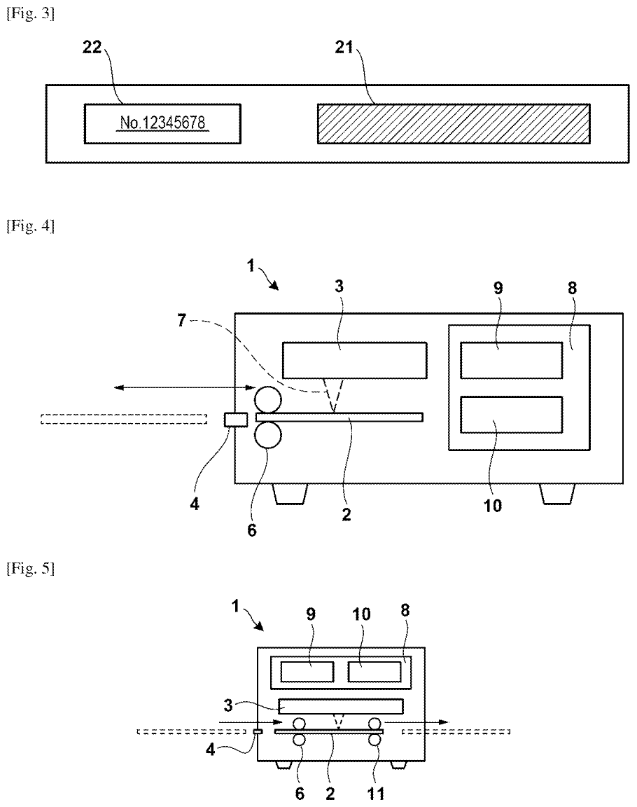

FIG. 4 is a cross-sectional view illustrating the measurement apparatus 1 according to one embodiment. Note that the reference sign 2 in FIG. 4 indicates a CI that has been conveyed to a measurement position of the measurement apparatus 1, and the dotted line indicates a CI before being inserted into the measurement apparatus 1. The CI that has been inserted into an insertion portion 4 of the measurement apparatus 1 is conveyed to the measurement position by a conveyance roller pair 6. A measurement unit 3 measures the color of the discoloration area 21 of a CI. After the measurement of a CI, the CI is discharged outside the measurement apparatus 1 by rotating the conveyance roller pair 6 in a reverse direction. The measurement unit 3 reads an optical measurement value relating to the color value of the discoloration area 21 of a CI, e.g. spectral reflectance, using light 7. In the present embodiment, the measurement unit 3 is configured to read the identification information of a CI in addition to the color value of the discoloration area 21 of a CI. If the identification portion 22 includes a serial number or a bar code, the measurement unit 3 can optically read the identification portion 22 of a CI. Note that if the identification portion 22 is an IC tag, the measurement unit 3 has an IC reader, and reads the identification information stored in the IC tag by communicating with the IC tag. Also, the measurement unit 3 can be configured to read the color value of the discoloration area 21 and the identification information by capturing an image of the discoloration area 21 and the identification portion 22 using an image sensor. A controller 8 controls the measurement apparatus 1. Various types of information to be used in the measurement are stored in a storage unit 9 of the controller 8. Also, a computational processing unit 10 of the controller 8 determines the color value of the discoloration area 21 of a CI based on a measurement result of the measurement unit 3, that is, spectral reflectance, for example. The controller 8 outputs the color information indicating the determined color value and the read identification information of a CI to a PC 15.

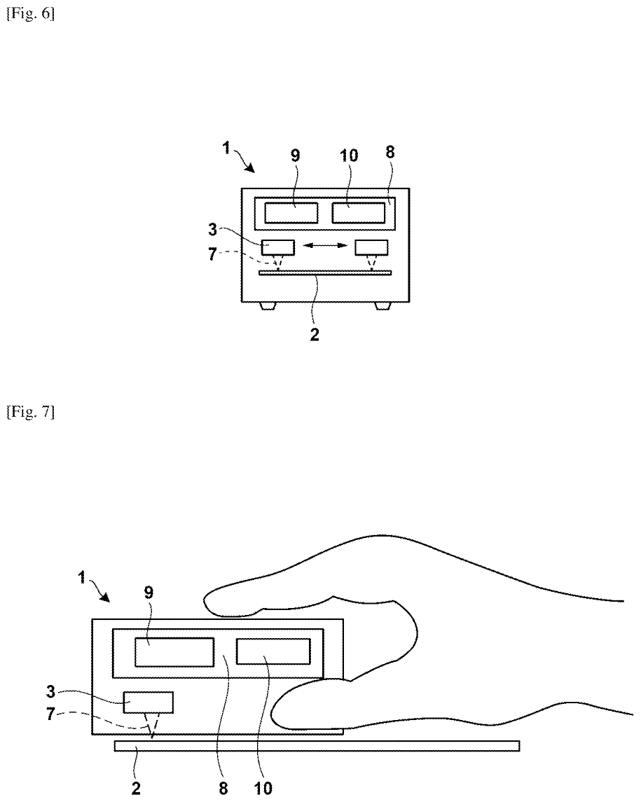

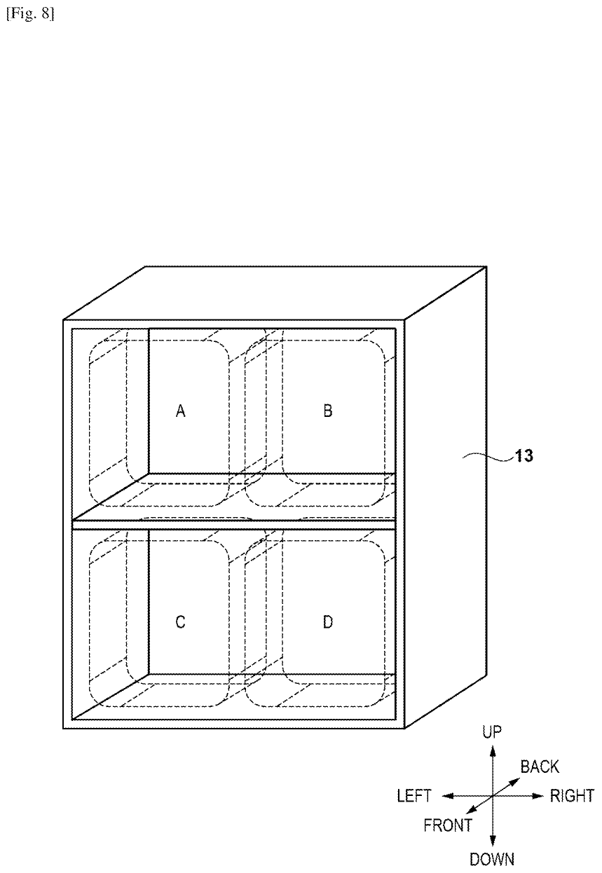

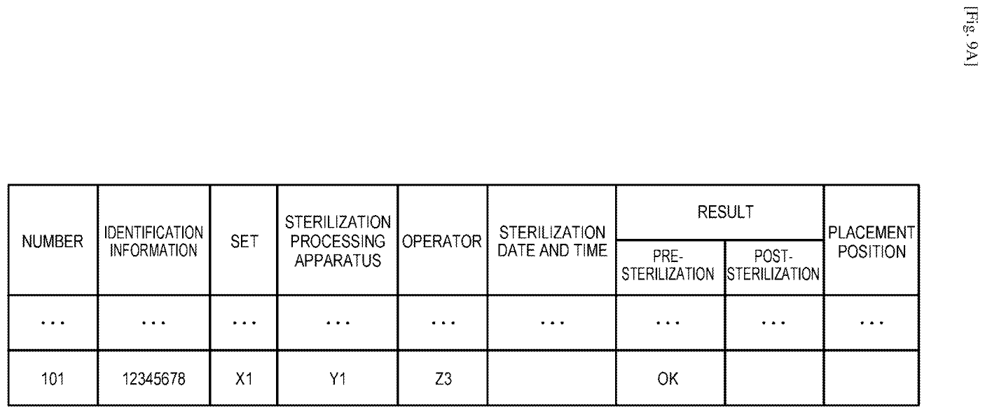

Note that the measurement apparatus 1 shown in FIG. 4 discharges a CI from the insertion portion 4, but the configuration of the measurement apparatus 1 may be such that, as shown in FIG. 5, a discharging portion is provided on an opposite side of the insertion portion 4 relative to the measurement position of a CI, and the CI is discharged from the discharging portion after measurement. Also, the measurement apparatus 1 shown in FIG. 4 is configured to measure, after a CI has been conveyed to the measurement position, the CI by a fixed measurement unit 3, but the measurement apparatus 1 may be configured such that, as shown in FIG. 6, the measurement unit 3 can be moved. That is, the configuration may also be such that the discoloration area 21 and the identification portion 22 of a CI are read by moving the measurement unit 3 in a predetermined direction. Moreover, as shown in FIG. 7, the configuration may also be such that the discoloration area 21 and the identification portion 22 of a CI are read by an operator moving the measurement apparatus 1 on the CI. Such a configuration is advantageous when CIs having various sizes are used.

Note that, when the identification information of a CI is not used as the identification information of a set, the identification information is read in accordance with a medium in which the identification information is recorded and a format of the identification information, and the identification information is output to a PC 15. For example, if the identification information is a bar code, a bar-code reader can be used for reading the identification information. Also, if the identification information is a serial number, a configuration may also be adopted in which an operator inputs the identification information to the management system 100.

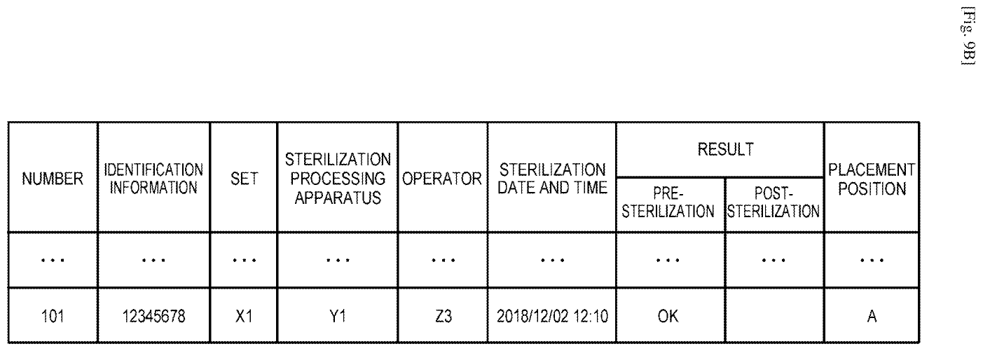

FIG. 8 shows the inside of the sterilization processing apparatus 13. In the present embodiment, a plurality of sub-regions are defined by diving a placement region in which sets are placed in the sterilization processing apparatus 13. Note that, in the following description, individual sub-regions are expressed as zones. In FIG. 8, four zones A to D are defined by dividing the placement region of sets into two in each of an up and down direction and a left and right direction. Note that the placement region of the sterilization processing apparatus 13 can be divided in any way and the number of zones is not limited to four. For example, the placement region can further be divided in a front and rear direction. Note that, in order for an operator to visually recognize the boundaries of zones inside the sterilization processing apparatus 13, partitions indicating the boundaries of zones can be provided inside the sterilization processing apparatus 13, or marks can be provided with tape or the like.

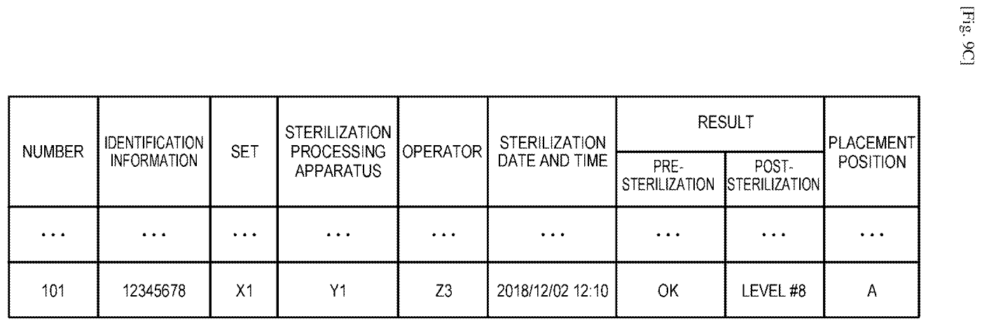

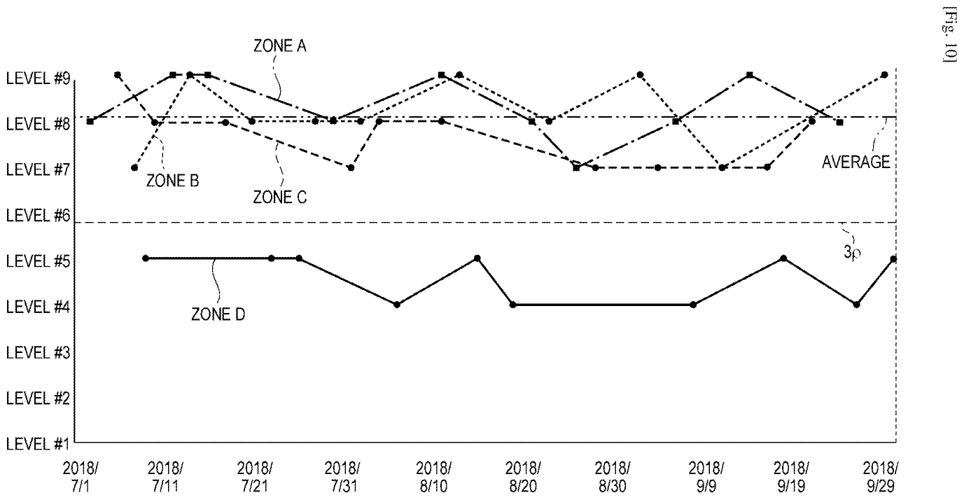

FIGS. 9A to 9C show a management information table (sterilization record table), which is management information retained in the server apparatus 14 in the present embodiment. A number field shows a number of each record. The identification information of a set is stored in an identification information field of the record. In the present embodiment, the identification information of a set is identification information of a CI enclosed in the set. In each record, sterilization related information related to a sterilization process performed on a set corresponding to the identification information stored in the identification information field is recorded. Hereinafter, the sterilization related information will be described. First, information for specifying the type of a corresponding set is stored in a set field. Information for specifying the sterilization processing apparatus 13 that performs sterilization of the set is stored in a sterilization processing apparatus field. Information for specifying an operator that performs the sterilization process on a set is stored in an operator field. Information indicating date and time at which the sterilization process has been performed is stored in a sterilization data and time field. A result information field includes two subfields for pre-sterilization and post-sterilization. In the present embodiment, the discoloration area 21 of a CI before the CI being enclosed in a set is measured by the measurement apparatus 1, and whether or not the CI has an initial failure is determined based on the measurement result. Also, the determination result is stored in the pre-sterilization subfield. Also, after the sterilization process of the set, the discoloration area 21 of a CI enclosed in the set is measured by the measurement apparatus 1, and the level of the achievement degree of the sterilization process is determined based on its measurement result. Also, the determined level of the achievement degree is stored in the post-sterilization subfield. Information indicating the zone inside the sterilization processing apparatus 13 in which a set is placed when the set is subjected to the sterilization process is stored in a placement position field.

For example, it is assumed that a CI whose identification information is 12345678, shown in FIG. 3, is enclosed in the packaging stage. An operator adds a new record (number 101 in FIG. 9A) to the management information table as a recording target record by operating a PC 15. Also, the operator inputs, to the management system 100, information for specifying the type of a target set of the packaging stage, information for specifying the sterilization processing apparatus to be used, and information for specifying the operator. With this, these pieces of information are respectively recorded in the set field, the sterilization processing apparatus field, and the operator field of the recording target record. The operator causes the measurement apparatus 1 to read the enclosed CI whose identification information is 12345678. With this, the measurement apparatus 1 outputs a color value (hereinafter, measured color value) Ct of the discoloration area 21, as a measurement result, and the identification information to the PC 15. The PC 15, after confirming that the record corresponding to the identification information 12345678 is not present in the management information table, records the identification information 12345678 in the identification information field of the recording target record.

Also, the PC 15 determines whether or not the color difference between the color value C #1, which is an initial color of the discoloration area 21 of the CI, and a measured color value Ct exceeds a threshold value. If the color difference between the color value C #1 and the measured color value Ct does not exceed the threshold value, the PC 15 records “OK” in the pre-sterilization subfield. FIG. 9A shows this state. Note that if the color difference between the color value C #1 and the measured color value Ct exceeds the threshold value, the PC 15 records “NG” in the pre-sterilization subfield. At the same time, the PC 15 performs display indicating whether or not the color difference between the color value C #1 and the measured color value Ct exceeds the threshold value. Also, if the color difference between the color value C #1 and the measured color value Ct exceeds the threshold value, a fact that the CI cannot be used in later stages can also be displayed in a display unit. The operator determines, based on the information displayed in the display unit of the PC 15, whether the measured CI is to be enclosed in a set, or the CI is discarded and a different CI is to be measured.

Next, the operator places a set in the sterilization processing apparatus 13, in the sterilization process. At this time, the operator inputs the zone of the sterilization processing apparatus 13 at which the set was placed to the management system 100. With this, the management system 100 records the information to the placement position field of a record corresponding to the set. Thereafter, the operator performs the sterilization process on the set by operating the sterilization processing apparatus 13. The management system 100 acquires completion date and time of the sterilization process from the sterilization processing apparatus 13, and records the acquired date and time to the sterilization data and time field of the record corresponding to the set. FIG. 9B shows this state. Thereafter, when the set is used in the operation stage, the discoloration area 21 of a CI enclosed in the set is measured by the measurement apparatus 1. With this, the measurement apparatus 1 outputs, to the PC 15, the measured color value Ct of the discoloration area 21 and the identification information 12345678, as the measurement result. The PC 15 determines the level of the achievement degree of the sterilization process based on the measured color value Ct, and records the determined level to the post-sterilization subfield of the record corresponding to the identification information 12345678. FIG. 9C shows this state.

Note that the configuration of obtaining information regarding the zone at which a set has been placed is not limited to a configuration in which an operator confirms the zone and input it to the management system 100. For example, the configuration may be such that a sensor provided inside the sterilization processing apparatus 13 detects the zone at which a set has been placed in the sterilization processing apparatus 13, and notifies the management system 100 of the zone. Alternatively, the configuration may also be such that, after a set is placed in the sterilization processing apparatus 13, an image sensor such as a camera captures an image of the placement state, and causes the PC 15 to read the captured image and determine the zone using an image recognition technique.

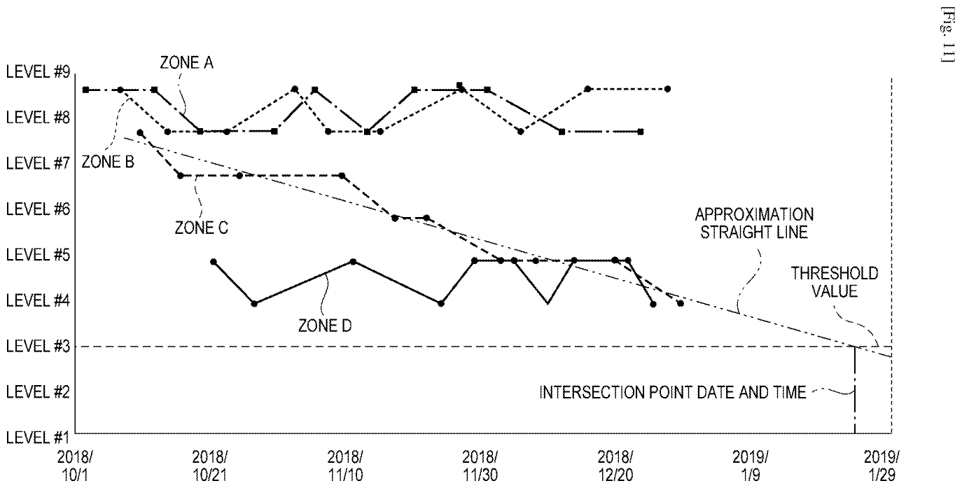

FIG. 10 shows an example of a graph created based on the management information. The management system 100 can present the graph shown in FIG. 10 to a user. FIG. 10 shows the change in level indicated by the post-sterilization fields of records whose values in the set field and the sterilization processing apparatus field are respectively X1 and Y1, with respect to the respective zones at which the sets have been placed. Note that the horizontal axis shows the value in the sterilization data and time field. The straight two-dot chain line in FIG. 10 indicates an average of the levels indicated by points in zones A to C excluding a zone D. Also, the straight broken line in FIG. 10 indicates a level obtained by subtracting an amount of three times of the standard deviation of the levels indicated by the points in zones A to C excluding a zone D from the average. It can be understood that, in FIG. 10, the achievement degrees of the sterilization process when sets of type X1 are placed in the zone D are separated from the standard data distribution. It can be understood that, from FIG. 10, when a set of type X1 is subjected to the sterilization process by the sterilization processing apparatus 13, the set should be placed in any of the zones A. B, and C, while avoiding locating the set in the zone D.

Therefore, if an operator has input, to the management system 100, a fact that a set of type X1 is placed in the zone D of the sterilization processing apparatus 13 specified by Y1 in the sterilization stage, the management system 100 can output a warning. The warning can include a message saying that a set of type X1 cannot be placed in the zone D. Moreover, the management system 100 can present, to a user, the zones at which a set of type X1 can be placed instead of, or in addition to the message saying that a set of type X1 cannot be placed in the zone D. Note that the combination of a set and a zone that cannot be used is determined based on predetermined criteria. For example, the management system 100 obtains an average value, a minimum value, and another statistic value of the achievement degree with respect to combinations of the types of set and the zones, and can determine that a combination whose average value, minimum value, or other statistic value is less than a threshold value is a combination that cannot be used. Note that records in a predetermined period in the past or records of a predetermined number in the past can be used to determine combinations that cannot be used.

Note that, if the level of the achievement degree of the sterilization process is low in a specific zone such as the zone D regardless of the type of a set, the management system 100 determines that the sterilization processing apparatus 13 specified by Y1 has a problem, and can prompt a user to adjust/repair the sterilization processing apparatus 13 specified by Y1. Note that the straight broken line in FIG. 10 is set based on an amount of three times of the standard deviation, but the multiplication factor to be applied to the standard deviation can be arbitrarily determined according to the probability desired to be obtained.

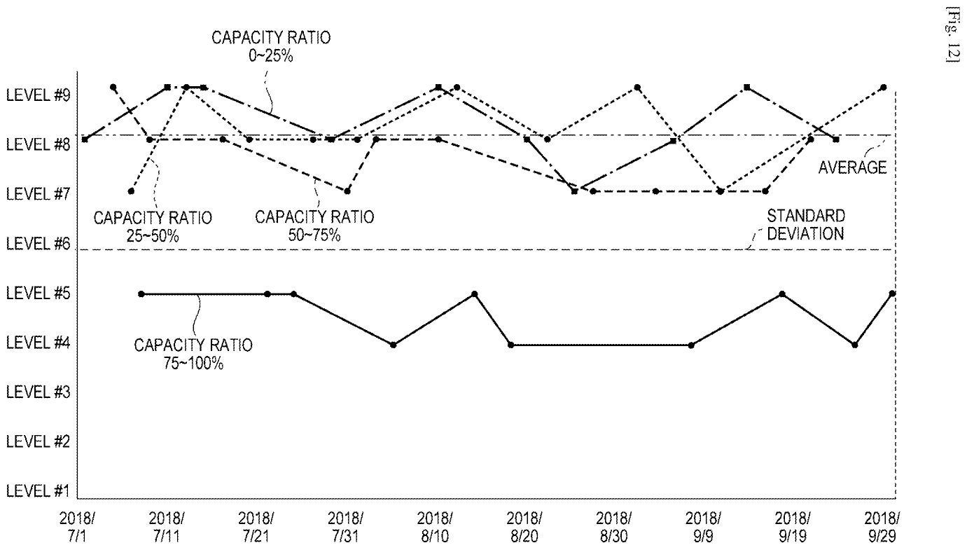

FIG. 11 shows a graph similar to that in FIG. 10. The management system 100 can present the graph shown in FIG. 1I to a user, for example. The approximation straight line indicated by the two-dot chain line in FIG. 11 is a straight line obtained by subjecting points indicating the change in the level in the zone C to linear approximation. The approximation straight line can be obtained using various statistical methods such as a least squares method. Note that, in the present embodiment, the change in the level in the zone C is approximated by a linear function, but any relational expression can be used, and an approximation curve can be obtained instead of the approximation straight line. It can be understood that, from the approximation straight line shown in FIG. 11, the achievement degree of the sterilization process performed on sets placed in the zone C decreases over time. Also, it can be determined what the level of the sterilization process will be in the future if the current trend continues by extending the approximation straight line to a future point in time from the current time. For example, in FIG. 11, it can be estimated that the level of the sterilization process performed on sets placed in the zone C reaches a level #3 in average around the date and time corresponding to the intersection point between the approximation straight line and a straight line (threshold value) corresponding to a threshold value, a level #3 being the threshold value. In this case, the management system 100 can present the estimated date and time to the user. Therefore, if it is estimated that, although the NG level has not been reached at the current time, there is a risk of “NG” being determined in the future, the user can take a measure such as adjusting/repairing the sterilization processing apparatus 13 before “NG” being determined.

Note that, in the present embodiment, a plurality of zones are defined by dividing the placement region of the sterilization processing apparatus 13, and information indicating the zone is recorded in the management information table. However, the configuration, in general, may be such that position information indicating the placement position at which a set has been placed inside the placement region is recorded in the management table. For example, coordinates at a predetermined resolution are defined inside the placement region, and the coordinates of the position of the placed set are recorded in the management table as the position information. Note that this also corresponds to the size of the zones being decreased relative to the size of a set.

As described above, when the sterilization process is performed, the position information indicating the placement position at which a sterilization target is placed is recorded. For example, as described in FIG. 8, the position information may indicate the zone. With this, the management system 100 can generate information for determining the relationship between the placement position and the achievement degree of the sterilization process. Also, information for determining the change in the achievement degree of the sterilization process at each placement position can be generated by recording information regarding sterilization date and time. Moreover, determination information for determining the relationship between the placement position and the achievement degree of the sterilization process can be created for each type of the set by recording information indicating the type of the set. Furthermore, determination information for determining the relationship between the placement position and the achievement degree of the sterilization process can be created for each combination of the type of the set and the sterilization processing apparatus by recording information for specifying the sterilization processing apparatus that has performed the sterilization process.

Note that, in the embodiment described above, information for specifying the sterilization processing apparatus 13 is recorded in the management information table, but instead thereof or in addition thereto, information for specifying the type of the sterilization processing apparatus 13 can also be recorded. With this, determination information for determining the relationship between the placement position and the achievement degree of the sterilization process can be generated for each of the combinations of the types of set and the types of sterilization processing apparatuses. Therefore, a measure can be taken such that a set is avoided from being placed at a placement position at which the achievement degree of the sterilization process performed on the set is low, for example. Also, whether or not adjustment/repair of the sterilization processing apparatus 13 is needed can also be determined.

Moreover, the management system 100 can determine the combinations of the types of set and the placement positions that are not to be used based on the determination information. The combinations that are not to be used may be combinations of the types of sets, the sterilization processing apparatuses, and the placement positions, or combinations of the types of sets, the types of the sterilization processing apparatuses, and the placement positions. If a combination determined not to be used is input to the management information, the management system 100 can give a warning to the user. The warning may include a message saying that the input combination cannot be used or a message for notifying the user of a combination that can be used. With the configuration described above, the quality of the sterilization process can be kept at a high level.

### Second Embodiment

Next, a second embodiment will be described focusing on the difference from the first embodiment. In the present embodiment, a capacity ratio field is provided in place of the placement position field shown in FIGS. 9A to 9C. The capacity ratio is a ratio of the total volume of all the sets placed in the sterilization processing apparatus 13 for subjecting to the sterilization process relative to the capacity of the placement region of the sterilization processing apparatus 13. For example, the total volume of the sets placed in the sterilization processing apparatus 13 can be calculated based on the number of sets, for each type, that are placed in the sterilization processing apparatus 13, by obtaining volumes of the respective types in advance. The management system records, in the capacity ratio field of a record, the capacity ratio when the set corresponding to the record is subjected to the sterilization process in the sterilization processing apparatus 13.

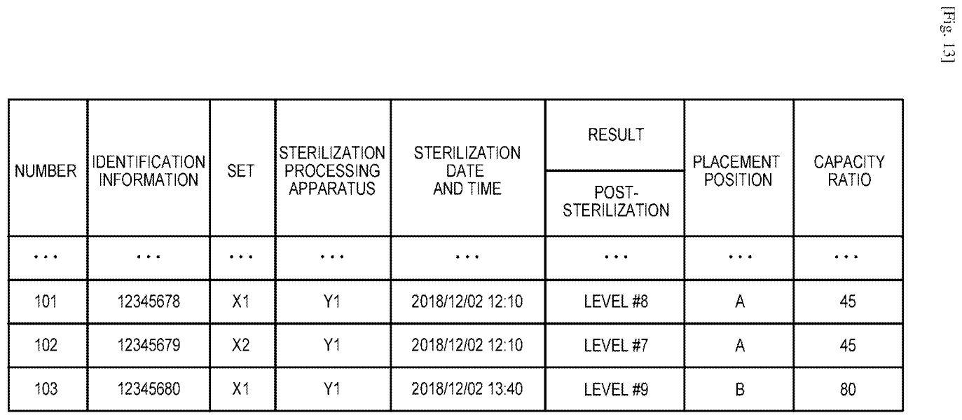

FIG. 12 shows an example of a graph created based on the management information of the present embodiment. The management system 100 can present the graph shown in FIG. 12 to a user, for example. FIG. 12 shows the change in the level indicated by the post-sterilization fields of records whose values in the set field and the sterilization processing apparatus field are respectively X1 and Y1 for each range of the capacity ratio. Note that the horizontal axis shows the value in the sterilization data and time field. The straight two-dot chain line in FIG. 12 indicates an average of levels indicated by points in the capacity ratio from 0 to 75%. Also, the straight broken line in FIG. 12 indicates a level obtained by subtracting an amount of three times of the standard deviation of the levels indicated by the points in the capacity ratio from 0 to 75% from the average. In FIG. 12, it can be understood that the achievement degree of the sterilization process performed at the capacity ratio of 75% or more is in a range separated from a standard data distribution. From FIG. 12, it can be understood that, when a set of type X1 is subjected to the sterilization process in the sterilization processing apparatus 13 specified by Y1, the sterilization process should be performed at a capacity ratio less than 75%. Also, when a set of type X1 is subjected to the sterilization process in the sterilization processing apparatus 13 specified by Y1, if an operator has input a value that is 75% or more in the capacity ratio field, the management system 100 can present a warning to the user. This warning can include a message, to the user, indicating that, when a set of type X1 is subjected to the sterilization process in the sterilization processing apparatus 13 specified by Y1, the capacity ratio should be less than 75%. Note that the straight broken line in FIG. 12 is set based on an amount of three times of the standard deviation, but the multiplication factor to be applied to the standard deviation can be arbitrarily determined according to the obtained probability.

As described above, information indicating the capacity ratio when the sterilization process is performed is recorded. With this, information for determining the relationship between the capacity ratio and the achievement degree of the sterilization process can be created. Note that, similarly to the first embodiment, determination information can also be generated for determining the change in achievement degree for each range of the capacity ratio, and for determining the relationship between the capacity ratio and the achievement degree of the sterilization process for each type of the set, and for each individual or type of the sterilization processing apparatus 13. Therefore, a set with respect to which the achievement degree of the sterilization process is not affected even if the capacity ratio is high, a set with respect to which the achievement degree of the sterilization process decreases as the capacity ratio increases, and the like can be determined.

Also, similarly to the first embodiment, the management system 100 can determine the combination, which will not be used, between the type of the set and the capacity ratio based on the determination information. The combination may be a combination of the type of the set, the capacity ratio, and the sterilization processing apparatus, or a combination of the type of the set, the capacity ratio, and the type of the sterilization processing apparatus. The management system 100, if a combination that has been determined not to be used is input to the management information, can give a warning to the user. The warning may include a message saying that the input combination cannot be used or a message for notifying the user of a combination that can be used. In this way, sets that are to be sterilized at the same time in the sterilization processing apparatus 13 can be selected according to the type of the set in order to keep the achievement degree of the sterilization process at a high level, and the quality of the sterilization process can be kept at a high level. Note that the configuration may also be such that a user inputs the upper limit value of the capacity ratio to the management system 100. The upper limit value may be a value recommended by a manufacturer of the sterilization processing apparatus, for example. Also, if a capacity ratio that exceeds the upper limit value is input to the capacity ratio field, the management system 100 can give a warning to the user. The warning may include a message notifying the user of a fact that the input capacity ratio exceeds the upper limit value.

### Third Embodiment

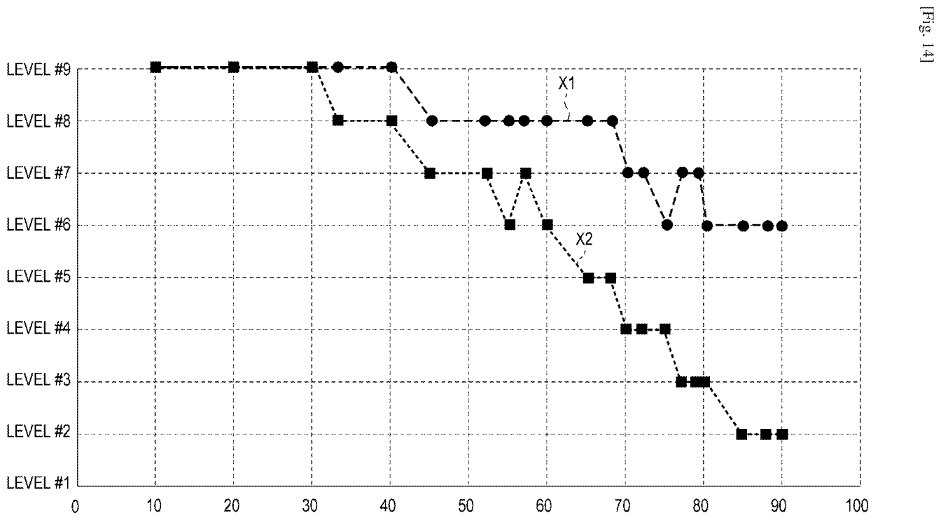

Next, a third embodiment will be described focusing on the difference from the second embodiment. FIG. 13 shows a management information table of the present embodiment. In the present embodiment, the management information table includes both of the placement position field and the capacity ratio field, and both of the zone in which a set has been placed and the capacity ratio in the sterilization process are recorded.

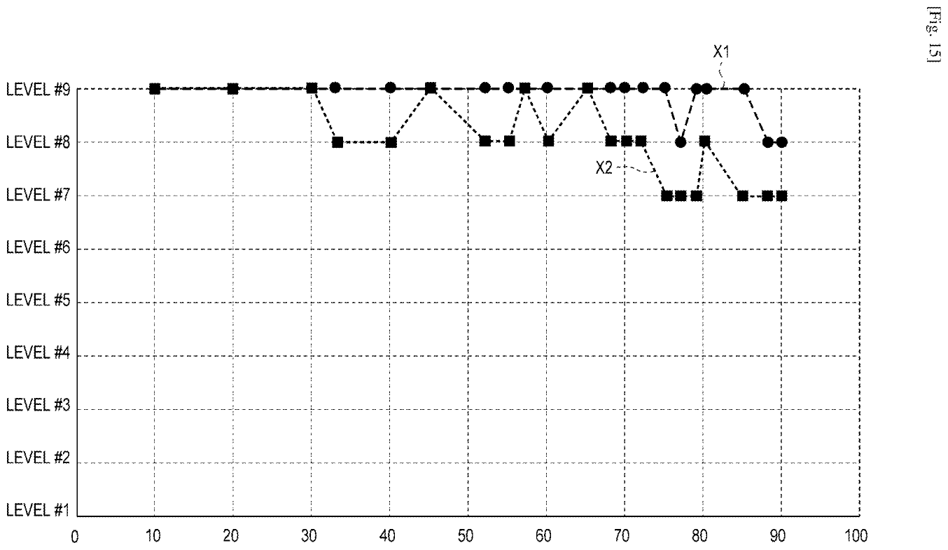

FIGS. 14 and 15 are examples of graphs created based on management information of the present embodiment. The management system 100 can present the graphs shown in FIGS. 14 and 15 to a user, for example. In FIGS. 14 and 15, the horizontal axis shows the capacity ratio, and the vertical axis shows the level of the achievement degree of the sterilization process. Note that FIG. 14 shows a graph illustrating the relationship between the capacity ratio of sets placed in the zone A and the level of the achievement degree for each type of the set. FIG. 15 shows a graph illustrating the relationship between the capacity ratio of sets placed in the zone B and the level of the achievement degree for each type of the set. From FIG. 15, it can be understood that, in the zone B, the achievement degree of the sterilization process is kept at a high level regardless of the capacity ratio. On the other hand, it can be understood that, in the zone A, the achievement degree of the sterilization process performed on sets of type X2 greatly decreases as the capacity ratio increases. From the graphs in FIGS. 14 and 15, it can be understood that, when sets of types X1 and X2 are sterilized by the sterilization processing apparatus 13 at the same time, sets of type X1 should be placed in the zone A, and sets of type X2 should be placed in the zone B. Also, from FIG. 15, when the capacity ratio is greater than or equal to a predetermined value, e.g. 50%, if the user has input that sets of type X2 are placed in the zone A, for example, the management system 100 can notify the user of a fact that the sets of type X2 should be placed in the zone B, or the sets of type X2 should not be placed in the zone A.

As described above, both of the placement position at which a sterilization target is placed and the capacity ratio are recorded. With this, determination information can be generated for determining the relationship between the combination of the placement position and the capacity ratio and the achievement degree of the sterilization process. Moreover, similarly to the embodiments described above, information can be generated for determining the relationship between the combination of the placement position and the capacity ratio and the achievement degree of the sterilization process for each type of the set, and for each individual or type of the sterilization processing apparatus 13.

Also, similarly to the first and second embodiments, the management system 100 can determine the combination, which will not be used, between the type of the set, the placement position, and the capacity ratio based on the determination information. The combination may also be a combination that further includes the sterilization processing apparatus or the type of the sterilization processing apparatus. If a combination that has been determined not to be used is input to the management information, the management system 100 can give a warning to the user. The warning may include a message saying that the input combination cannot be used or a message for notifying the user of a combination that can be used. With the configuration described above, a combination that may degrade the quality of the sterilization process can be avoided, and the quality of the sterilization process can be kept at a high level.

### Fourth Embodiment

Next, a fourth embodiment will be described focusing on the difference from the embodiments described above. In general, a CI is also placed at a spot (hereinafter, a cold spot) that is the most difficult to be sterilized inside the sterilization processing apparatus 13 in addition to a CI to be enclosed in a set in order to confirm whether the sterilization process has been properly performed by the sterilization processing apparatus 13. In the following, the CI that is not enclosed in a set and is placed inside the sterilization processing apparatus 13 is expressed as a PCD CI. When the sterilization process by the sterilization processing apparatus 13 is completed, the PCD CI is measured by the measurement apparatus 1, and the achievement degree of the sterilization process is determined based on a measured color value Ct of a discoloration area 21 of the PCD CI. Also, determination of an initial failure is also performed on the PCD CI.

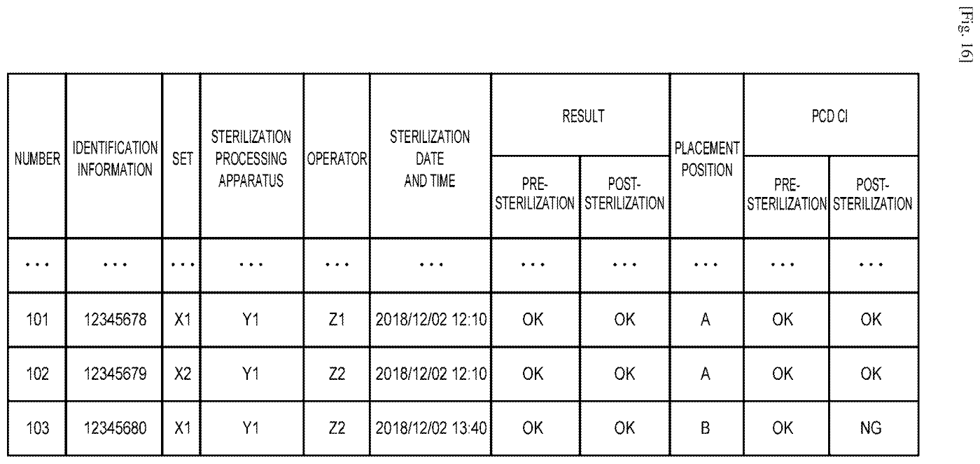

In the present embodiment, the PCD CI is placed in each zone in addition to the cold spot. Also, in the management information, the determination result of an initial failure of the PCD CI placed in the same zone, and the determination result of the achievement degree determined based on the PCD CI are also recorded, as shown in FIG. 16. Note that, in FIG. 16, instead of recording the level of the achievement degree, whether the achievement degree is “OK” or “NG” is recorded. If the determination result of the achievement degree determined from the PCD CI placed in a certain zone and the determination result of the achievement degree determined from the CI of a set placed in this zone are both “OK”, it can be determined that the sterilization process has no problem, for example. On the other hand, if, although the determination result based on the PCD CI is “OK”, the determination result based on the CI of a set is “NG”, it can be understood that the sterilization condition outside the packaging member is satisfied, but the sterilization condition inside the packaging member is not satisfied. In this case, it can be estimated that there is a problem in the assembling of the set. Moreover, if both of the determination result based on the PCD CI and the determination result determined based on the CI of a set are “NG”, it can be understood that the sterilization condition outside the packaging member is not satisfied. In this case, it can be estimated that the sterilization processing apparatus 13 has failed, or there is a problem in overloading of sets, or the like.

Note that, in the present embodiment, the PCD CI is placed in each zone, but a configuration may also be such that the PCD CI is placed in at least one of the plurality of zones. In this case, the determination result of the PCD CI in a zone, of zones in which PCD CIs are placed, that is closest to the zone in which the set corresponding to a record is placed is recorded to the record corresponding to the set. More generally, the determination result of a PCD CI placed closest to the placement position of a set, is recorded in a record corresponding to the set.

As described above, one or more PCD Cis are placed inside the placement region, and the achievement degrees determined by the PCD CIs are recorded. The management system 100 compares the achievement degree based on the CI and the achievement degree based on the PCD CI of the same record, and present the comparison result to a user. Accordingly, whether or not there is a problem in the assembling or packaging of a set can be determined, and a necessary measure can be taken. Therefore, the quality of the sterilization process can be kept at a high level.

### Fifth Embodiment

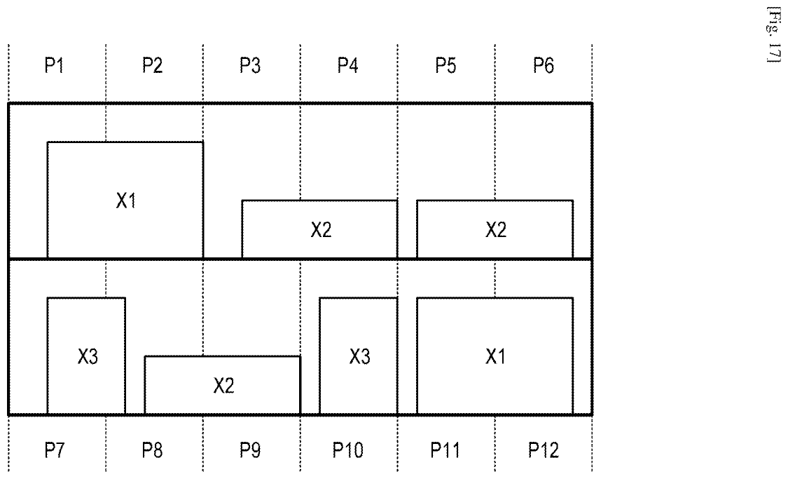

Next, a fifth embodiment will be described focusing on the difference from the first embodiment. In the present embodiment as well, similarly to the first embodiment, a plurality of sub-regions (zones) are defined by dividing a placement region in which sets are placed in the sterilization processing apparatus 13. Note that the size of each zone is set such that a plurality of sets cannot be contained in one zone. FIG. 17 shows a state in which upper and lower placement regions are each divided in six zones, that is, the placement region is divided into a total or 12 zones, zones P1 to P12.

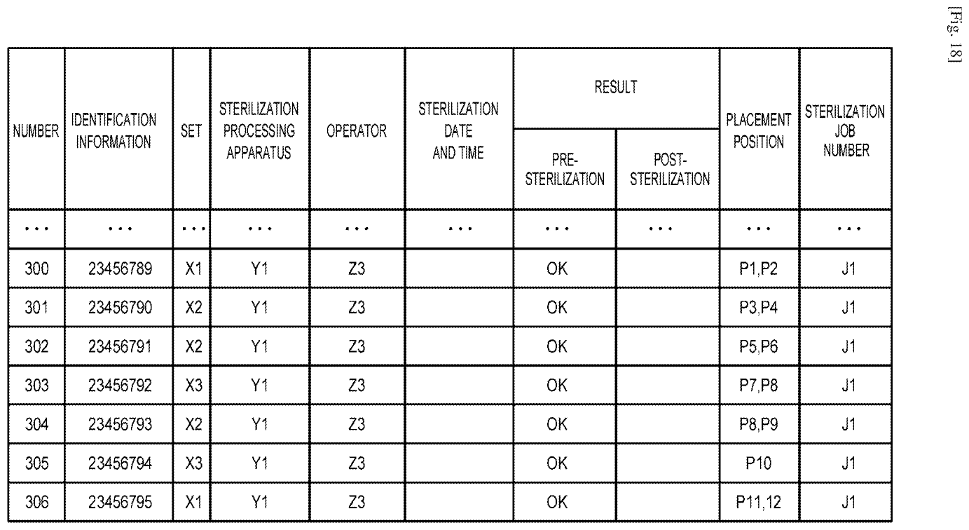

FIG. 18 shows a management information table (sterilization record table), which is management information retained in the server apparatus 14, in the present embodiment. Information indicating the zone inside the sterilization processing apparatus 13 in which a set is placed when the set is subjected to the sterilization process is stored in a placement position field. Note that with respect to a set placed over a plurality of zones, all of the zones over which the set is placed are recorded. The records of numbers from 300 to 306 in FIG. 18 respectively correspond to records of the seven sets in FIG. 17. For example, an upper left set of type X1 is placed in the zones P1 and P2, and corresponds the record of number 300. Also, a lower left set of type X3 is placed in zones P7 and P8, and corresponds to the record of number 303.

Also, in the present embodiment a sterilization job number field is provided in the management information table. Also, the same value is stored in the sterilization job number fields of sets that are subjected to the sterilization process by the same one sterilization processing apparatus 13 at the same time. The management system 100 can specify the sets that have been subjected to the sterilization process by the same sterilization processing apparatus 13 at the same time from the values in the sterilization job number field.

The management system 100 can specify, with respect to a plurality of sets that have been subjected to the sterilization process at the same time, which sets are in an adjacent relationship from values in the placement position field. Specifically, from the values in the placement position field in FIG. 18, the management system 100 can specify that the set of type X1 placed over zones P1 and P2 and the set of type X2 placed over zones P3 and P4 are in an adjacent relationship. The same applies to the other adjacent relationships.

The management system 100 determines the level of a pair of sets that are placed adjacent to each other. In the present embodiment, the level of a pair is defined as the smaller one of the achievement levels of the sterilization process of the two sets of the pair. For example, it is assumed that, in FIG. 18, a level #8 is stored in the post-sterilization subfield of a record of number 300, and a level #6 is stored in the post-sterilization subfield of a record of number 301. In this case, the level of the pair of the set of type X1 placed over zones P1 and P2 and the set of type X2 placed over zones P3 and P4 is a level #6.

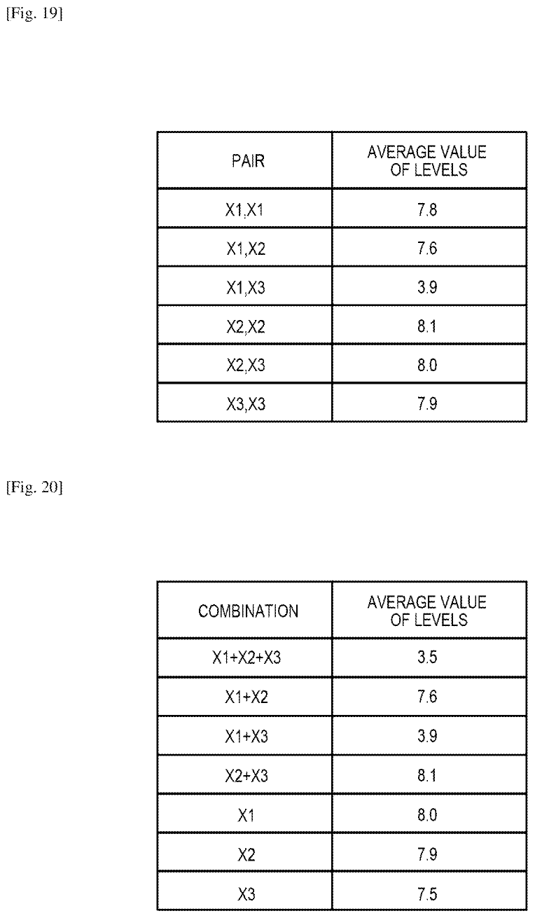

The management system 100 can present, with respect to each pair, a graph similar to that illustrated in FIGS. 10 and 11 in the first embodiment to a user. Also, the management system 100 can obtain an average value of levels of a pair. Note that the records in a predetermined period in the past or records of a predetermined number in the past can be used to obtain the average value. FIG. 19 shows average values of levels of respective pairs. As shown in FIG. 19, it can be understood that the average value of levels of the pair of type X1 and type X3 is lower than the average values of levels of the other pairs. For example, the management system 100 can determine the pair whose average value is smaller than a predetermined value (threshold value) is a pair not to be used. Also, if a pair not to be used is input to the management information, the management system 100 can give a warning to an operator. For example, if the threshold value is 4, the management system 100 determines that the sets of type X1 and type X3 cannot be placed adjacent to each other. When an operator makes an input to a record having the same sterilization job number, the management system 100 determines the adjacent relationship in the sterilization processing apparatus 13 based on the values input to the set fields and the placement position fields. Also, if sets of type X1 and type X3 are placed adjacent to each other, the management system 100 presents a warning indicating that the sets of type X1 and type X3 cannot be placed adjacent to each other to a user.

Note that, in the present embodiment, a case where a set of type X1 is present on a right side of a set of type X2 is not distinguished from a case where a set of type X1 is present on a left side of a set of type X2, and both cases are treated as a pair of sets of type X1 and type X2. However, a case where a set of type X1 is present on a right side of a set of type X2 and a case where a set of type X1 is present on a left side of a set of type X2 can also be treated as different pairs. Also, the level of a pair is defined as the smaller one of levels of two sets included in the pair, but the level of a pair may be an average value of two levels. Also, the management system 100 determines the adjacent relationship based on values in the placement position fields. That is, the information of the placement position field in the present embodiment is information for specifying the adjacent relationship. However, the configuration may be such that the adjacent relationship is specified by recording identification information of a neighboring set to the record. Moreover, the present invention is not limited to determining a pair not to be used based on the average value of levels of sets of the pair, and the determination can be made using any statistic values.

As described above, in the present embodiment, the determination information for determining the relationship between the adjacent relationship of types of sets and the level can be generated and presented to the user. Note that, similarly to the first embodiment, the determination information for determining the relationship between the adjacent relationship and the achievement degree of the sterilization process can also be generated for each individual or type of the sterilization processing apparatus 13. Also, similarly to the first embodiment, the management system 100 can determine the adjacent relationship not to be used based on the determination information. The adjacent relationship not to be used can also be determined for each sterilization processing apparatus or for each type of the sterilization processing apparatus. If an operator places sets in an adjacent relationship not to be used, the management system 100 can give a warning to the operator. With this configuration, the quality of the sterilization process can be kept at a high level.

### Sixth Embodiment

Next, a sixth embodiment will be described focusing on the difference from the fifth embodiment. In the present embodiment, similarly to the fifth embodiment, a sterilization job number field is provided in the management information table (sterilization record table). Note that the placement position field may be omitted.

The management system 100 can specify the combination of types of sets that have been subjected to the sterilization process at the same time from the values in the sterilization job number fields. Specifically, the management system 100 can specify a fact that a combination of types X1, X2, and X3 has been subjected to the sterilization process at the same time from the values in the sterilization job number fields in FIG. 18. That is, the information stored in the sterilization job number field is information for specifying sets that have been subjected to the sterilization process by the same sterilization processing apparatus 13 at the same time.

The management system 100 determines the level of the combination that has been subjected to the sterilization process at the same time. In the present embodiment, the level of a combination is defined as the smallest one of levels of the respective sets that have been subjected to the sterilization process at the same time. For example, if the minimum value in the post-sterilization subfield of records of numbers 300 to 306, in FIG. 18, is a level #3, the level of the combination of sets shown in FIG. 17 is a level #3.

The management system 100 can present, with respect to each combination, a graph similar to that illustrated in FIGS. 10 and 11 in the first embodiment to a user. Also, the management system 100 can obtain, for each combination, an average value of the levels. Note that the records in a predetermined period in the past or records of a predetermined number in the past can be used to obtain the average value. FIG. 20 shows average values of levels of respective combinations. From FIG. 20, it can be understood that the average value of levels decreases if the types X1 and X3 are included in the combination. For example, the management system 100 determines a combination whose average value is smaller than a predetermined value (threshold value), and can determine that such a combination is a combination that is not to be used. Also, when information is input to a record, the management system 100 determines the combination of sets to be subjected to the sterilization process based on the values stored in the sterilization job number field. Also, if this combination is included in the combinations not to be used, the management system 100 can give a warning to an operator. Specifically, in a case shown in FIG. 20, the management system 100 can give a warning to a user if sets of types X1 and X3 are included in a combination.

Note that, in the present embodiment, even if a plurality of sets of the same type are included, the number thereof is not taken into consideration. However, a combination in which the number of sets is considered can also be adopted. In this case, the combination in FIG. 17 is a combination of two sets of type X1, three sets of type X2, and two sets of type X3. This corresponds to determining, not the combination of types of sets, the combination of sets. Also, the management system 100 determines the combination of sets that are to be sterilized at the same time based on the values in the sterilization job number field. That is, the information of the sterilization job number field of the present embodiment is information for specifying the combination. However, the configuration may also be such that the combination is specified by recording pieces of identification information of sets to be sterilized at the same time to each record. Moreover, the present invention is not limited to determining a combination not to be used based on the average value of levels of the sets included in the combination, and the determination can be made using any statistic values.

As described above, in the present embodiment, the relationship between the combination of sets to be subjected to the sterilization process at the same time and the level can be presented to a user. Also, the management system 100 determines the combination with respect to which the level decreases, and if an operator places sets of such a combination, can give a warning to an operator. Note that the combination may also be a combination including a sterilization processing apparatus or the type of a sterilization processing apparatus. With this configuration, the quality of the sterilization process can be kept at a high level.

### Seventh Embodiment

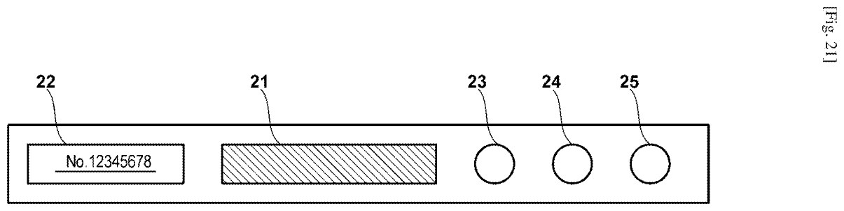

Next, a seventh embodiment will be described focusing on the difference from the first to sixth embodiments. In the present embodiment, a CI shown in FIG. 21 is used. The CI shown in FIG. 21 includes temperature detection regions 23 to 25 that discolor according to temperature, in addition to a discoloration area 21 and an identification portion 22. For example, the temperature detection region 23 is a region that discolor when the temperature is at 125 degrees centigrade or more, the temperature detection region 24 is a region that discolor when the temperature is at 130 degrees centigrade or more, and the temperature detection region 25 is a region that discolor when the temperature is at 135 degrees centigrade or more, for example. Therefore, if the temperature inside the sterilization processing apparatus 13 does not reach 125 degrees centigrade, the color of any of the temperature detection regions 23 to 25 does not change. Also, if the temperature inside the sterilization processing apparatus 13 is 125 degrees centigrade or more and less than 130 degrees centigrade, only the color of the temperature detection region 23 changes. Also, if the temperature inside the sterilization processing apparatus 13 is 130 degrees centigrade or more and less than 135 degrees centigrade, the colors of the temperature detection regions 23 and 24 change, and the color of the temperature detection region 25 does not change. Moreover, if the temperature inside the sterilization processing apparatus 13 is 135 degrees centigrade or more, all of the colors of the temperature detection regions 23 to 25 change. That is, as a result of using the CI shown in FIG. 21, a highest temperature in the sterilization process by the sterilization processing apparatus 13 can be detected. Note that the number of temperature detection regions is not limited to three.

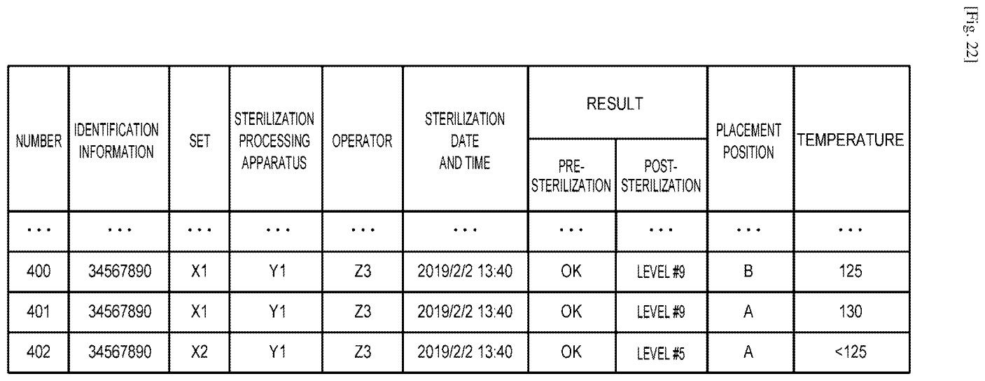

FIG. 22 shows a management information table (sterilization record table), which is management information retained in the server apparatus 14, in the present embodiment. According to FIG. 22, a “temperature” field is added to the management information table of the first embodiment shown in FIG. 9. A detection result of the temperature determined using the temperature detection regions 23 to 25 of a CI is recorded in the temperature field. Note that the determination of temperature can be made by the measurement apparatus 1 measuring the colors of the temperature detection regions 23 to 25. According to FIG. 22, the record of number 400 indicates that the temperature is 125 degrees centigrade or more and less than 130 degrees centigrade. Also, the record of number 402 indicates that the temperature is less than 125 degrees centigrade.

The management system 100 can generate information for determining the change in temperature for each zone based on the management information table in FIG. 22. Also, if the temperature is less than or equal to a threshold value, the sterilization process is affected, and therefore a configuration may be adopted in which the management system 100 presents a warning to a user when the temperature is less than or equal to the threshold value. Also, similarly to the method illustrated in FIG. 1I, the date and time at which the temperature becomes less than or equal to the threshold value can be estimated, and the estimated date and time can be presented to a user.

Note that, in the present embodiment, the change in temperature for each zone is determined, but the configuration can be such that, by combining the method with that of the second embodiment, the change in temperature for each range of the capacity ratio is determined. Moreover, the configuration can be such that, by combining the method with that of the third embodiment, the change in temperature for each combination of the range of the capacity ratio and the zone. Furthermore, by combining the methods of the fifth and sixth embodiments, the temperature can be determined for each adjacent relationship or combination. Also, each temperature detection region of the CI can also be configured such that the color changes if a predetermined temperature continues for a predetermined time or more.

Also, a density detection region for detecting the density of a sterilizing agent may be provided in place of or in addition to the temperature detection region 25. More generally, a configuration can be adopted in which the CI is provided with a detection region for detecting one or more predetermined physical quantities, and the relationship between the zones or the like and the one or more predetermined physical quantities is determined.

### Eighth Embodiment

Next, an eighth embodiment will be described focusing on the difference from the fifth to seventh embodiments. The positions of a sterilization target and a CI inside a packaging member and the relative positional relationship between the sterilization target and the CI may affect the discoloration of the discoloration area 21 of the CI. Therefore, in the present embodiment, the placement regions of the sterilization target and the CI inside a packaging member are also recorded (placement region information) in the management information table. Note that, similarly to the zones in the sterilization processing apparatus, the inside of the packaging member is divided into a plurality of regions, and the region inside the packaging member at which the CI is placed and the regions inside the packaging member at which one or more sterilization targets are placed are recorded. Note that, with respect to each of the one or more sterilization targets, all of the regions at which the sterilization target is placed are recorded.

According to the configuration described above, the management system 100 can generate information for determining the relationship between the combination of the placement position of a set inside a sterilization processing apparatus and the placement region of a CI and/or a sterilization target inside of a packaging member and the achievement degree of the sterilization process. Also, as a result of recording information of the sterilization date and time, information for determining the change in achievement degree of the sterilization process can be generated for each combination. Moreover, as a result of recording information indicating the type of a set, determination information for determining the relationship between the combination and the achievement degree of the sterilization process can be generated for each type of the set. Furthermore, as a result of recording information for specifying the sterilization processing apparatus that has performed the sterilization process, determination information for determining the relationship, for each combination of the type of the set and the sterilization processing apparatus, between the combination and the achievement degree of the sterilization process can be generated. As a result of presenting the determination information to a user, the quality of the sterilization process can be kept at a high level.

### Ninth Embodiment

Next, a ninth embodiment will be described focusing on the difference from the eighth embodiment. In the present embodiment as well, similarly to the eighth embodiment, the placement regions of a CI and/or a sterilization target in a packaging member are recorded in the management information table. Also, in the present embodiment, information for determining the relationship between the placement region of the CI inside the packaging member and the achievement degree is generated. Alternatively, information for determining the relationship between the combination of the placement regions of the CI and the sterilization target inside the packaging member and the achievement degree is generated. Moreover, similarly to the first embodiment and the like, for any of each type of the set, each sterilization processing apparatus, and each type of the sterilization processing apparatus, or for each of any combinations thereof, information for determining the relationship between the placement regions of the CI and the sterilization target and the achievement degree can be generated. Note that the information to be generated can be similar to the information in which the zones in which the set is placed in the first embodiment is replaced by the placement regions of the CI or the combinations of the placement regions of the CI and the sterilization target, for example.

Note that when the type of a packaging member differs for each type of a set, the type of the set can be associated with the type of the packaging member. However, there are cases where packaging members of the same type are used to sets of different types. In this case, as a result of recording the type of the packaging member to the management information table, the information for determining, for each type of the packaging member, the relationship between the placement regions of the CI and the sterilization target and the achievement degree can be generated.

Also, the management system 100 can determine the region at which the CI should be placed in the packaging member based on the relationship between the placement region of the CI inside the packaging member and the achievement degree, for example. The region at which the CI should be placed can be determined for any of each type of the set, each sterilization processing apparatus, and each type of the sterilization processing apparatus, or for each of any combinations thereof. Also, the type of the set can also be replaced by the type of the packaging member. Note that the region at which the CI should be placed can be determined similarly to the determination of a placement position not to be used in the first embodiment. Note that, in the present embodiment, different from the first embodiment, it is determined that the CI should be placed at a region inside the packaging member at which the determined achievement degree is smallest or smaller than a threshold value determined based on the relationship between the placement region of the CI and the achievement degree. This is to prevent the achievement degree of the sterilization process on a set from being over-evaluated. Also, the management system 100 can determine the region at which the sterilization target should be placed in the packaging member based on the relationship between the placement region of the sterilization target inside the packaging member and the achievement degree, for example. Note that the region inside the packaging member at which the sterilization target should be placed is a region inside the packaging member at which the determined achievement degree is high.

Also, the management system 100 can give a warning to a user if the placement region of the CI or the sterilization target in the packaging member that is input to the management information is a region different from the region at which the CI or the sterilization target should be placed, similarly to the embodiments described above. The warning may include a message notifying the user of the region at which the CI or the sterilization target should be placed. With the configuration described above, the quality of the sterilization process can be kept at a high level.

Functional Block of Management System

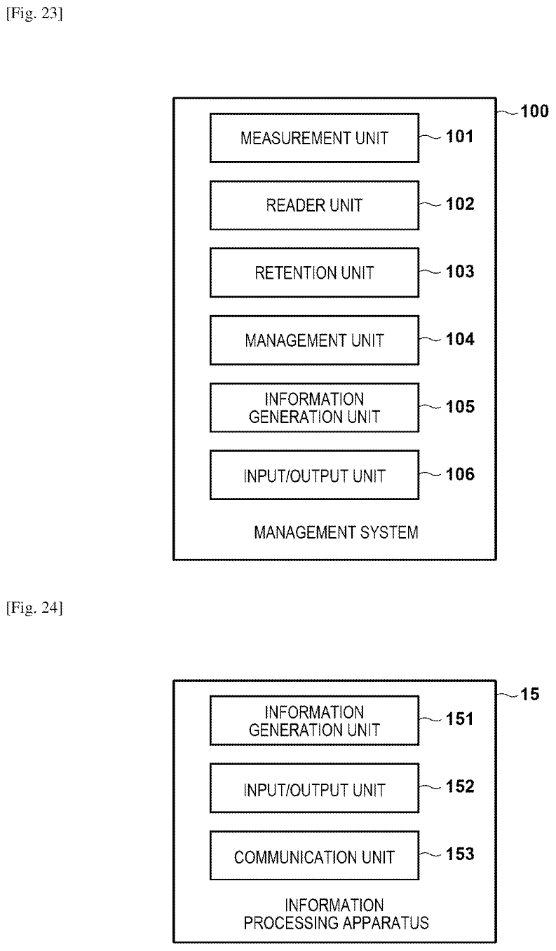

FIG. 23 is a functional block diagram of the management system 100 shown in FIG. 2. An input/output unit 106 corresponds to the PCs 15, for example, accepts an input of information that a user records to the management information, and outputs information to be presented to the user. A measurement unit 101 corresponds to the measurement apparatuses 1 and the PCs 15, and measures the color value of the discoloration area 21 of a CI. A reader unit 102 corresponds to the measurement apparatuses 1 and the PCs 15, and reads identification information of a CI, which is identification information of a set. Note that the present invention is not limited to the configuration in which each measurement apparatus 1 reads the identification information of a set, as described above. For example, when identification information is attached to the packaging member of the set without using the identification information of the CI as the set identifier, for example, an IC reader or a bar-code reader can be used as the reader unit 102. Moreover, when the identification information is attached to the packaging member of a set, the configuration may also be such that, without providing the reader unit 102, the user inputs the identification information of the set using the input/output unit 106. A retention unit 103 corresponds to a storage unit such as a hard disk of the server apparatus 14, and retains the management information, which is sterilization record information. The management unit 104 corresponds to the processors of the server apparatus 14 and the PCs 15, and performs addition of information to the management information, update of the management information, deletion of unnecessary information recorded into the management information as needed, and the like, based on a user operation or the like. The information generation unit 105 corresponds to the processors of the server apparatus 14 and the PCs 15, and generates various types of information as described above based on the sterilization record information and presents the generated information to the user via the input/output unit 106. Also, the information generation unit 105 presents various warnings to the user.

Functional Block of Information Processing Apparatus

Note that each PC 15 in the management system in FIG. 1 is also an information processing apparatus that generates various types of information based on the sterilization record information and presents the generated information to the user. FIG. 24 is a functional block diagram of the PC 15 (information processing apparatus). An input/output unit 152 corresponds to a keyboard, a mouse, a display, a printer, and a storage apparatus of the PC 15, receives operational inputs made by the user, and outputs information to be presented to the user. A communication unit 153 is an accessing unit for acquiring sterilization record information retained by the server apparatus 14 by accessing the server apparatus 14. Also, the information generation unit 151 generates various types of information as described above based on the acquired sterilization record information, and presents the generated information to the user via the input/output unit 152. Also, the information generation unit 151 presents various warnings to the user.

### Other Embodiments

Note that, in the first embodiment, a mode of recording the position information indicating the placement position of a set in the placement region of the sterilization processing apparatus 13 is described, and in the second embodiment, a mode of recording the capacity ratio of the placement region is described. Also, in the fifth and sixth embodiments, a mode of recording the adjacent relationship, and the combination of sets or types of the sets to be placed in the sterilization processing apparatus 13 is described. The present invention is not limited to the modes of recording these pieces of information, and may be a mode of recording any placement information relating to the placement of sets to the sterilization processing apparatus 13. That is, the placement position in the first embodiment, the capacity ratio in the second embodiment, the adjacent relationship in the fifth embodiment, information for specifying the combination of sets or the types of the sets in the sixth embodiment are each an example of the placement information. Also, the management system 100 generates information for determining the relationship between the placement information and the achievement degree.

Also, in the embodiments described above, the measurement apparatuses 1 are connected to the respective PCs 15, the PCs 15 each determine the level of the sterilization process based on the color value of the discoloration area 21 acquired from the connected measurement apparatus 1, and record the determination result to the management information retained by the server apparatus 14. However, the configuration may also be such that the level of the sterilization process is determined in each measurement apparatus 1. In this case, a configuration may be adopted in which the measurement apparatuses 1 are connected to the network, and the measurement apparatuses 1 each transmit the determined level of the sterilization process to the server apparatus 14. Moreover, the configuration may also be such that the determination result is displayed in an unshown display unit of each measurement apparatus 1 instead of connecting the measurement apparatus 1 to the network. In this case, an operator inputs determination result displayed in the display unit of each measurement apparatus 1 to the management system 100.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-034745, filed on Feb. 27, 2019 and Japanese Patent Application No. 2019-131401, filed on Jul. 16, 2019 which are hereby incorporated by reference herein in their entirety.

1. A management system that manages records of sterilization processes performed by a sterilization processing apparatus on a set including one or more sterilization targets and a first indicator including a discoloration area whose color changes according to an achievement degree of a sterilization process, the management system comprising:
retaining means for retaining sterilization record information including the achievement degree of the sterilization process determined based on a color of the discoloration area of the first indicator of the set, and placement information regarding placement of the set to the sterilization processing apparatus, with respect to each sterilization process performed on the set; and
generating means for generating information for determining a relationship between the placement information and the achievement degree of the sterilization process based on the sterilization record information.

2. The management system according to claim 1,
wherein the placement information includes position information indicating a placement position at which the set is placed in the sterilization processing apparatus, and
the generating means generates information for determining a relationship between the placement position indicated by the position information and the achievement degree of the sterilization process, based on the sterilization record information.

3. The management system according to claim 2,
wherein the sterilization record information further includes information indicating a type of the set with respect to the each sterilization process performed on the set, and
the generating means generates determination information for determining a relationship between the placement position and the achievement degree of the sterilization process for each type of the set.

4. The management system according to claim 3, further comprising warning means for determining a combination, of a type of the set and the placement position, that will not be used based on the determination information generated by the generating means, and giving a warning to a user if the combination that will not be used is input to the sterilization record information.

5. The management system according to claim 3,
wherein the sterilization record information further includes information indicating the sterilization processing apparatus that has performed the sterilization process or a type of the sterilization processing apparatus with respect to the each sterilization process performed on the set, and
the determination information is information for each combination of the type of the set and the sterilization processing apparatus, or each combination of the type of the set and the type of the sterilization processing apparatus.

6. The management system according to claim 5, further comprising warning means for determining a combination, of the type of the set, the sterilization processing apparatus, and the placement position, that will not be used, or a combination, of the type of the set, the type of the sterilization processing apparatus, and the placement position, that will not be used based on the determination information generated by the generating means, and giving a warning to a user if the combination that will not be used is input to the sterilization record information.

7. The management system according to any one of claims 2 to 6, wherein the sterilization record information further includes information indicating a date and a time at which the sterilization process has been performed with respect to the each sterilization process performed on the set, and
the generating means generates information for determining a change in the achievement degree of the sterilization process for the each placement position indicated by the position information.

8. The management system according to claim 2,
wherein the placement information further includes information indicating a ratio of a sum of volumes of respective sets placed in a placement region of the sterilization processing apparatus that has performed the sterilization process relative to a capacity of the placement region in which the sets are placed in the sterilization processing apparatus, and
the generating means generates information for determining a relationship between the achievement degree of the sterilization process and a combination of the placement position and the ratio.

9. The management system according to claim 8,
wherein the sterilization record information further includes information for indicating a type of the set, with respect to the each sterilization process performed on the set, and
the generating means generates, for each type of the set, determination information for determining a relationship between the achievement degree of the sterilization process and a combination of the placement position and the ratio.

10. The management system according to claim 9, further comprising warning means for determining a combination, of the type of the set, the placement position, and the ratio, that will not be used, based on the determination information generated by the generating means, and giving a warning to a user if the combination that will not be used is input to the sterilization record information.

11. The management system according to any one of claims 2 to 10, wherein a second indicator including a discoloration area whose color changes according to the achievement degree of the sterilization process is placed in the sterilization processing apparatus, and the sterilization process is performed, and
the sterilization record information further includes information indicating an achievement degree of the sterilization process based on a color of the discoloration area of the second indicator that is placed at a position closest to the placement position at which the set is placed, with respect to the each sterilization process performed on the set.

12. The management system according to claim 11, wherein the generating means generates a comparison result obtained by comparing the achievement degree of the sterilization process performed on the set, which is determined by the first indicator, and the achievement degree of the sterilization process performed on the same set, which is determined by the second indicator, the achievement degrees being included in the sterilization record information.

13. The management system according to any one of claims 2 to 12, wherein the position information indicates a sub-region of a plurality of sub-regions obtained by dividing a placement region in which the set is to be placed in the sterilization process.

14. The management system according to claim 1,
wherein the placement information includes information indicating a ratio of a sum of volumes of respective sets placed in a placement region of the sterilization processing apparatus relative to a capacity of the placement region in which the sets are placed in the sterilization processing apparatus that has performed the sterilization process, and the generating means generates information for determining a relationship between a range of the ratio and the achievement degree of the sterilization process based on the sterilization record information.

15. The management system according to claim 14,
wherein the sterilization record information further includes information indicating a type of the set with respect to the each sterilization process performed on the set, and
the generating means generates, for each type of the set, determination information for determining a relationship between the range of the ratio and the achievement degree of the sterilization process.

16. The management system according to claim 15, further comprising warning means for determining a combination, of the type of the set and the ratio, that will not be used based on the determination information generated by the generating means, and giving a warning to a user if the combination that will not be used is input to the sterilization record information.

17. The management system according to any one of claims 14 to 16, wherein the sterilization record information further includes information indicating a date and a time at which the sterilization process has been performed with respect to the each sterilization process performed on the set, and
the generating means generates information for determining a change in the achievement degree of the sterilization process for each range of the ratio.

18. The management system according to claim 1,
wherein the sterilization record information further includes information indicating a type of the set with respect to the each sterilization process performed on the set,
the placement information includes position information indicating a placement position at which the set is placed in the sterilization processing apparatus, and
the generating means generates determination information for determining a relationship between a pair of types of the sets placed adjacent to each other in the sterilization processing apparatus and the achievement degree of the sterilization process, based on the sterilization record information.

19. The management system according to claim 18, further comprising warning means for determining a pair of types of the sets that will not be places adjacent to each other in the sterilization processing apparatus based on the determination information generated by the generating means, determining pairs of types of the sets that are placed adjacent to each other in the sterilization processing apparatus based on types of the sets and the placement positions that are input to the sterilization record information, and giving a warning to a user if the pairs of types of the sets placed adjacent to each other in the sterilization processing apparatus include the pair of types of the sets that will not be placed adjacent to each other in the sterilization processing apparatus.

20. The management system according to claim 1,
wherein the placement information includes information for specifying a combination of the sets placed in the sterilization processing apparatus, and
the generating means generates determination information for determining a relationship between the combination of the sets placed in the sterilization processing apparatus and the achievement degree of the sterilization process, based on the sterilization record information.

21. The management system according to claim 20, further comprising warning means for determining a combination of the sets that will not be used based on the determination information generated by the generating means, and giving a warning to a user if the combination of the sets that will not be used is input to the sterilization record information.

22. The management system according to claim 20 or 21, wherein the sterilization record information further includes information indicating a type of the set, with respect to the each sterilization process performed on the set, and
a combination of the sets is a combination of types of the sets.

23. The management system according to any one of claims 1 to 22, wherein the sterilization record information includes information indicating a placement region of the first indicator inside the set, and the generating means generates information for determining a relationship between the achievement degree of the sterilization process and a combination of the placement information and the placement region of the first indicator.

24. The management system according to claim 23, wherein the sterilization record information includes information indicating placement regions of the one or more sterilization targets inside the set, and the generating means generates information for determining a relationship between the achievement degree of the sterilization process and a combination of the placement information, the placement regions of the first indicator and the one or more sterilization targets.

25. The management system according to any one of claims 1 to 24, wherein the first indicator includes a detection region whose color changes according to a physical quantity,
the sterilization record information includes a detection result of the physical quantity based on the detection region, and
the generating means generates information for determining the physical quantity for each placement information based on the sterilization record information.

26. The management system according to claim 25, wherein the physical quantity is temperature or density of a sterilizing agent to be used in the sterilization process.

27. A management system that manages records of sterilization processes performed by a sterilization processing apparatus on a set including one or more sterilization targets and a first indicator including a discoloration area whose color changes according to an achievement degree of the sterilization process, the management system comprising:
retaining means for retaining sterilization record information including the achievement degree of the sterilization process determined based on a color of the discoloration area of the first indicator of the set, and first placement region information indicating a placement region of the first indicator inside the set, with respect to the each sterilization process performed on the set; and
generating means for generating determination information for determining a relationship between the first placement region information and the achievement degree of the sterilization process based on the sterilization record information.

28. The management system according to claim 27,
wherein the sterilization record information includes second placement region information indicating placement regions of the one or more sterilization targets inside the set, and
the generating means generates determination information for determining a relationship between the achievement degree of the sterilization process and a combination of the first placement region information and the second placement region information.

29. The management system according to claim 27 or 28, further comprising means for determining a region at which the first indicator should be placed inside the set, based on the determination information.

30. An information processing apparatus that generates information based on records of sterilization processes performed by a sterilization processing apparatus on a set including one or more sterilization targets and a first indicator including a discoloration area whose color changes according to an achievement degree of the sterilization process, the information processing apparatus comprising:
accessing means for accessing an apparatus that retains sterilization record information including the achievement degree of the sterilization process determined based on a color of the discoloration area of the first indicator of the set, and placement information regarding placement of the set to the sterilization processing apparatus, with respect to the each sterilization process performed on the set; and
generating means for generating information for determining a relationship between the placement information and the achievement degree of the sterilization process based on the sterilization record information.

31. A computer-readable storage medium storing a program, the program causing a computer to function as the information processing apparatus according to claim 30.

32. A management method in a management system that manages records of sterilization processes performed by a sterilization processing apparatus on a set including one or more sterilization targets and a first indicator including a discoloration area whose color changes according to an achievement degree of the sterilization process, the management method comprising:
recording sterilization record information including the achievement degree of the sterilization process determined based on a color of the discoloration area of the first indicator of the set, and placement information regarding placement of the set to the sterilization processing apparatus, with respect to the each sterilization process performed on the set; and
generating information for determining a relationship between the placement information and the achievement degree of the sterilization process based on the sterilization record information.
